(19) United States
(12) Patent Application Publication
(10) Pub. No.: US 2014/0128577 A1
(43) Pub. Date: May 8, 2014

(54) PURIFICATION OF CHIMERIC PROTEIN

(71) Applicants: Samir Kulkarni, Mumbai (IN); Ravikant Devakate, Nanded (IN); Neeru Gupta, Ambala (IN); Prashant Kardekar, Indore (IN)

(72) Inventors: Samir Kulkarni, Mumbai (IN); Ravikant Devakate, Nanded (IN); Neeru Gupta, Ambala (IN); Prashant Kardekar, Indore (IN)

(73) Assignee: DR. REDDY'S LABORATORIES LIMITED, Qutubullapur (IN)

(21) Appl. No.: 14/124,440

(22) PCT Filed: Jun. 22, 2012
(86) PCT No.: PCT/IB2012/053160

§ 371 Date: Jan. 20, 2014

(30) Foreign Application Priority Data
Jun. 24, 2011 (IN) 2137/CHE/2011

(51) Int. Cl. C07K 16/00; C07K 14/715

(52) U.S. Cl. CPC C07K 16/00; C07K 14/7151
USPC 530/387.3

(57) ABSTRACT

Provided is a method for purification of TNFR:Fc fusion protein comprising hydrophobic interaction chromatography, wherein the buffer solution used in the said chromatography does not contain any additives.

### INTRODUCTION

Aspects of the present invention relate to a method of purification of TNFR: Fc fusion protein using chromatography techniques.

Tumor necrosis factor (TNF) is the dominant mediator of the cytokine cascade and plays a central role in inflammatory response. Elevated levels of TNF have been linked to many clinical conditions, including those associated with autoimmune disorders such as rheumatoid arthritis, ankylosing spondylitis and psoriasis. Recombinant TNFR:Fc fusion proteins (Tumor Necrosis Factor Receptor: Fc Fusion Protein) bind to the cytokine TNF and block the activity of TNF. Thus as potential inhibitors of TNF, they reduce the effect of TNF associated with these conditions.

An example of TNFR:Fc protein is Etanercept, a dimeric fusion protein consisting of the extracellular ligand-binding portion of the human 75 kilodalton (p75) tumor necrosis factor receptor (TNFR) linked to the Fc portion of human IgG1.

Etanercept has 934 amino acids with an apparent molecular weight of approximately 150 kilodaltons. The molecular structure of etanercept and its mechanism of action has been reviewed by Goffe B, Cather J C. (Journal of the American Academy of Dermatology, Volume 49, Issue 2, Supplement 1, August 2003, Pages 105-111). Being a fusion protein, Etanercept has greatly extended half-life in the bloodstream, and therefore, a more profound and long-lasting biologic effect. Etanercept is used in treatment of autoimmune diseases such as rheumatoid arthritis, ankylosing spondylitis, juvenile rheumatoid arthritis, psoriasis, psoriatic arthritis and potentially treat a variety of other disorders mediated by excess TNF.

Therapeutic TNFR:Fc fusion proteins, including Etanercept are produced by recombinant DNA technology. And, proteins expressed by recombinant DNA methods are typically associated with impurities such as host cell proteins (HCP), host cell DNA (HCD), viruses, etc. In addition, TNFR:Fc being a dimeric fusion protein, other impurities such as aggregates and variants are frequently formed and pose difficulty in the purification process. The presence of these impurities is a potential health risk, and hence their removal from the final product is a regulatory requirement and create a significant challenge in the development of methods for the purification of therapeutic proteins in general and TNFR:Fc in particular.

The prior-art discloses purification of fusion proteins by hydroxyapatite chromatography, hydrophobic interaction chromatography, ion exchange chromatography or affinity chromatography and or combinations thereof. U.S. Pat. No. 7,122,641, describes a method of purifying TNFR:Fc fusion protein using hydroxyapatite chromatography. US 2010256337 describes a method of purifying Fc containing proteins using blue-dye affinity chromatography and US20090306351 reports purification of proteins using Protein-A affinity chromatography. WO 2008025747 explains a method for purifying an Fc-fusion protein by affinity chromatography, cation exchange chromatography, anion exchange chromatography and hydroxyapatite chromatography.

Use of hydrophobic interaction chromatography in protein purification provides several advantages and simplicity over other chromatographic techniques. The advantages include working over a large temperature and pH range, ability to reduce non-specific protein binding, increased protein recovery, and effective removal of aggregates, protein A leachates and HCD. The prior-arts mentioned above do not disclose, hydrophobic interaction chromatography for the purification of TNFR:Fc fusion protein. However, hydrophobic interaction chromatography has been used for the purification of antibodies or proteins in general; U.S. Pat. No. 5,641,870 describes a process of purification of antibodies by hydrophobic interaction chromatography using a low pH (2.5-4.5) elution buffer, and U.S. Pat. No. 7,223,848 discloses a method of dissociating Fc-containing molecules from complexes of protein A/Fc-containing molecules by hydrophobic interaction chromatography using a low pH buffer of 4.1-4.5 containing arginine wherein the Fc-containing molecule is obtained in the flow-through.

U.S. Pat. No. 6,933,370 teaches a method of purifying highly anionic protein using a hydrophobic interaction chromatography column wherein the column is washed using a solution containing ethanol or isopropanol. U.S. Pat. No. 7,427,659 describes a method of separating a target protein by hydrophobic interaction chromatography operated in a flow-through mode wherein the impurities are bound to the column and target proteins are allowed to pass-through the column.

The methods described in the prior art involve either the use of a low pH and/or an organic solvent for elution of proteins. This may lead to denaturation and aggregation of the protein. In addition, the described prior-arts have stated the use of chaotropic agents or aggregation inhibitors to prevent such formation of aggregates. However, the use of chaotropes or aggregation inhibitors adds to the complexity of the downstream process. Given the therapeutic and commercial importance of TNFR:Fc proteins, an alternative process that alleviate the difficulties of prior-art is desirable. The principle object of the present invention is to provide a method for purification of TNFR:Fc by hydrophobic interaction chromatography with ease of operation that also avoids the use of additives such as organic solvents, low pH conditions, chaotropic agents or aggregate inhibitors.

### SUMMARY

Aspects of the present disclosure provide a method for the purification of TNFR:Fc fusion protein comprising a hydrophobic interaction chromatography employed in a bind elute mode and avoids the use of additives such as, organic solvents, low pH conditions or chaotropic agents or aggregate inhibitors in the purification process.

### BRIEF DESCRIPTION OF THE DRAWINGS

FIG. 1 Protein A Chromatogram obtained after performing purification as described in example 1 of the invention. Peak A represents the eluate obtained.

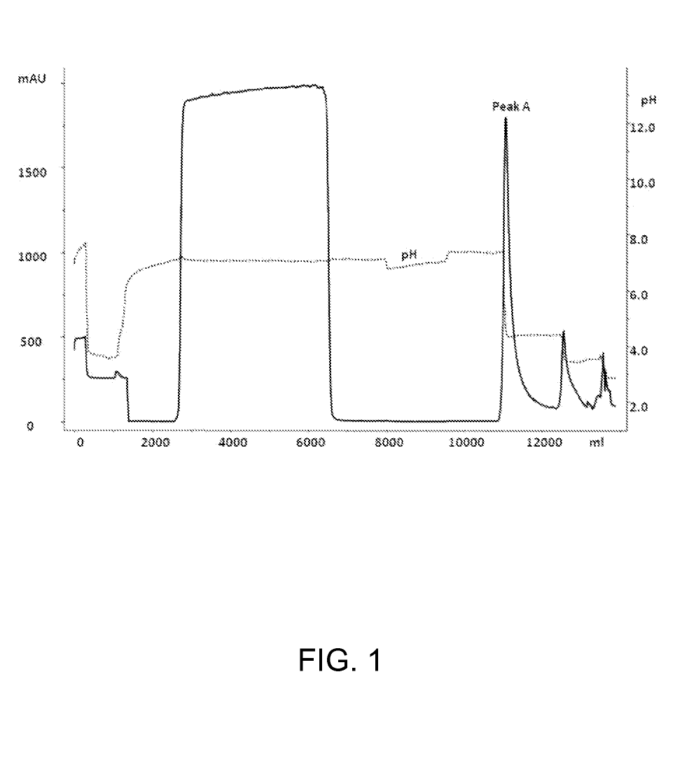

FIG. 2: Anion exchange Chromatogram obtained after performing purification as described in example 2 of the invention. Peak A represents the eluate obtained.

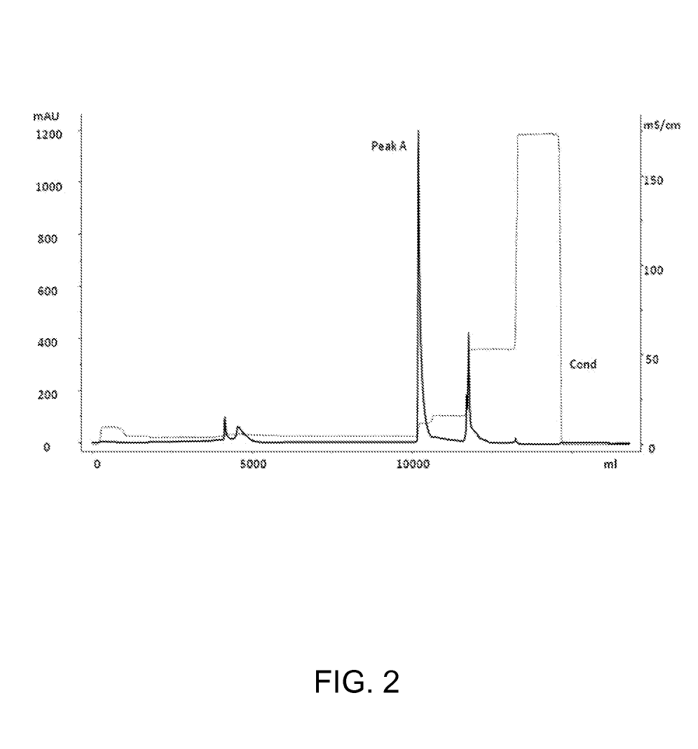

FIG. 3: Hydrophobic Interaction Chromatogram obtained after performing purification as described in example 3 of the invention. Peak A represents the eluate obtained.

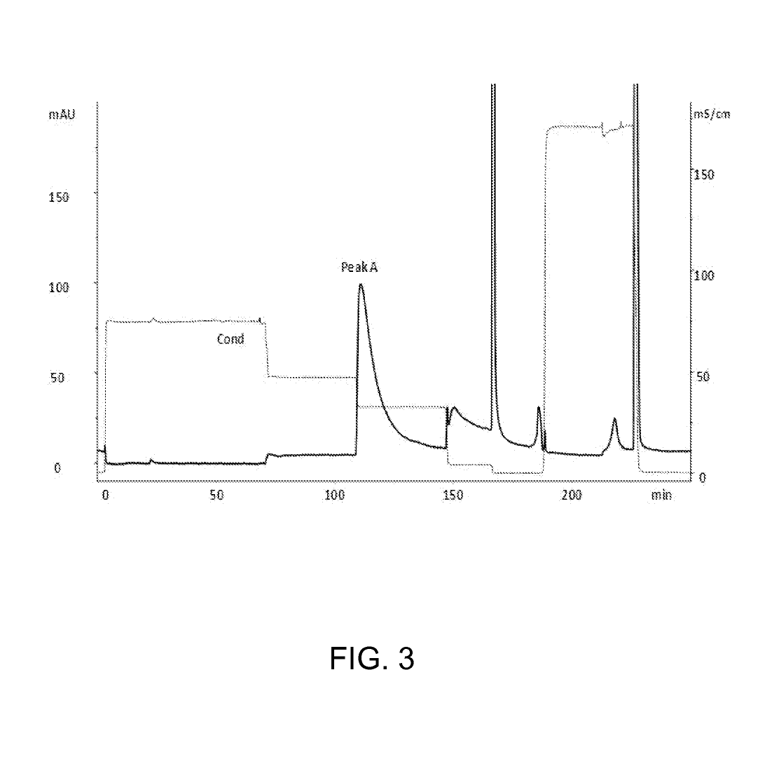

FIG. 4: Hydrophobic Interaction Chromatogram obtained after performing purification as described in example 4 of the invention. Peak A represents the eluate obtained.

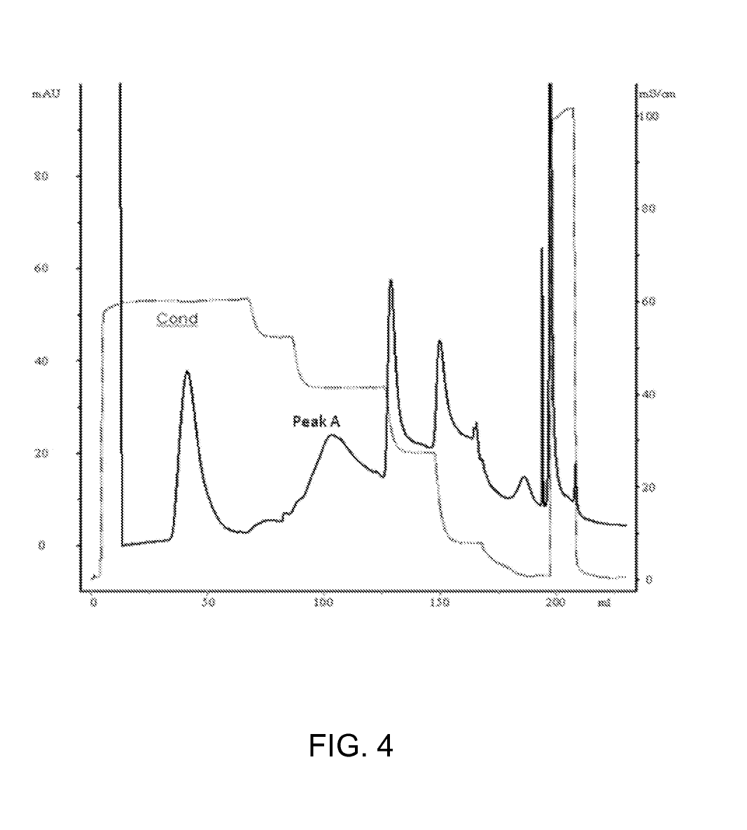

FIG. 5: Anion exchange Chromatogram obtained after performing purification as described in example 5 of the invention. Peak A represents the eluate obtained.

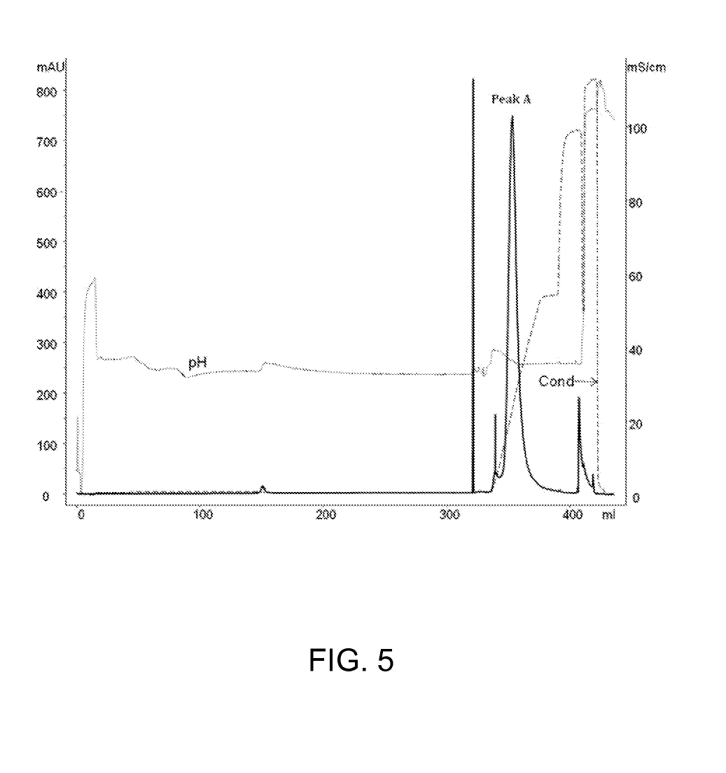

### DETAILED DESCRIPTION

The present invention describes a chromatographic process for purification of TNFR:Fc fusion proteins comprising a hydrophobic interaction chromatography performed in bind elute mode.

The term ‘bind elute mode’ as used herein refers to a mode of chromatographic purification, wherein the desired protein is bound to the chromatography resin when loaded and subsequently eluted with an elution buffer. The desired polypeptide may be collected as a single fraction or as various fractions.

The term ‘additives’ as used herein refers to a group comprising of, but not limited to organic solvents, chaotropic agents, aggregate inhibitors, solubilizing agents, amino acids; their derivatives or salts or variants or combinations thereof or substances which could perform similar function(s).

In embodiments, the invention provides a method for the purification of TNFR:Fc fusion protein comprising hydrophobic interaction chromatography performed in bind elute mode wherein the hydrophobic interaction chromatography step does not involve the use of additives either before, during, or after loading the mixture comprising TNFR:Fc.

In embodiments, the purification of TNFR:Fc fusion protein comprising a hydrophobic interaction chromatography wherein hydrophobic interaction chromatography is performed at a pH of about 6.0 to about 7.0. In yet another embodiment hydrophobic interaction chromatography may be preceded or followed by Anion exchange chromatography.

In an embodiment, the invention provides a method for the purification of TNFR:Fc fusion protein comprising steps of;

a) Protein-A chromatography

b) Hydrophobic Interaction chromatography and

c) Anion-exchange chromatography wherein the said hydrophobic interaction chromatography is performed in bind elute mode and the hydrophobic interaction chromatography step does not involve use of additives either before, during, or after loading the mixture comprising TNFR:Fc.

In embodiments, the invention provides a method for the purification of TNFR:Fc fusion protein comprising steps of;

a) Protein-A chromatography

b) Anion-exchange chromatography and

c) Hydrophobic Interaction chromatography

wherein the said hydrophobic interaction chromatography is performed in bind elute mode and the hydrophobic interaction chromatography step does not involve use of additives either before, during, or after loading the mixture comprising TNFR:Fc.

The protein A chromatographic resin used may be any protein A or variant or a functional fragment thereof coupled to any chromatographic support. Protein A chromatography mentioned in the embodiments may be carried out on columns that are available commercially including ProSep(R) controlled-pore glass resins produced by Millipore and MabSelect™, cross-linked agarose resin products produced by Amersham Biosciences, and other types of protein-A affinity chromatography resins, including gel-based resins and silica-based resins. In the embodiments above protein A resin used is Prosep VA ultra column (Millipore). For the purpose of the invention, fresh (i.e. not used before) protein A chromatographic resin may be used to obtain a feed stream for the second chromatographic step.

Hydrophobic interaction chromatography mentioned in the embodiments may be carried out on columns that are available commercially. These include, but are not limited to, SEPHAROSE columns such as Phenyl SEPHAROSE™ (Pharmacia LCK Biotechnology, AB, Sweden), FAST FLOW™ column with low or high substitution (Pharmacia LKB Biotechnology, AB, Sweden); Octyl SEPHAROSE High Performance column (Pharmacia LKB Biotechnology, AB, Sweden); FRACTOGEL™ EMD Propyl or FRACTOGEL, EMD Phenyl columns (E. Merck, Germany); MACRO- pREp™ Methyl or MACRO-PREPTM t-Butyl Supports (Bio-Rad, Calif.); WP HI-Propyl (C3) column (J. T. Baker, N.J.); and TOYOPEARL ether, phenyl or butyl columns (TosoHaas, Pa.). In embodiments of the application, a hydrophobic interaction chromatography resin, such as TSK-Butyl 650M (TOSOH Biosciences) is used. This resin is made of 1000 Å pore size base resin to which a butyl ligand is linked.

Anion exchange chromatography mentioned in the embodiments may be carried out using any commercially available anion exchange resins include, but are not limited to, DEAE cellulose, Poros PI 20, PI 50, HQ 10, HQ 20, HQ 50, D 50 from Applied Biosystems, MonoQ, MiniQ, Source 15Q and 30Q, Q, DEAE and ANX Sepharose Fast Flow, Q Sepharose high Performance, QAE SEPHADEX and FAST Q SEPHAROSE from GE Healthcare, Macro-Prep DEAE and Macro-Prep High Q from Biorad, Q-Ceramic Hyper D, DEAE-Ceramic Hyper D, from Pall Corporation. In embodiments of the application, a strong anion exchange resin, such as Q-Sepharose Fast Flow™ (GE Healthcare Life Sciences) is used. This resin is made using a highly cross-linked 6% agarose matrix attached to a —O—CH2CHOHCH2OCH2CHOHCH2—N+(CH3)3 functional group.

The chromatographic steps mentioned in the embodiment may include one or more tangential flow filtration, concentration, diafiltration or ultrafiltration steps.

The embodiments mentioned herein may include one or more viral inactivation steps or sterile filtration or nano filtration steps. The embodiments mentioned herein may include one or more neutralization steps.

The buffering agents used in the buffer solutions include, and are not limited to citrate, phosphate, hydrochloride, acetate, chloride, succinate, MES, MOPS, TRIS or ammonium and their salts or derivatives as well as combinations of these.

The invention is more fully understood by reference to the following examples. These examples should not, however, be construed as limiting the scope of the invention.

### EXAMPLE 1

### Chromatography I: Protein A Chromatography

The clarified cell culture broth was subjected to a protein A affinity chromatography to purify Etanercept. The protein A chromatography was carried out on a Prosep VA ultra column (Millipore). The cell culture supernatant was loaded onto the protein A chromatography column that was equilibrated with equilibration buffer, 50 mM Sodium acetate pH 7.0 and 0.15 M NaCl. The column was then washed with the same buffer followed by high salt wash with 50 mM Sodium acetate pH 7.0 with 0.75 M NaCl. The bound protein was eluted with 70% of 0.2 M Acetic acid and 30% 50 mM Sodium acetate.

### EXAMPLE 2

### Chromatography II: Anion Exchange Chromatography

The eluate from example 1 was loaded onto the anion exchange chromatographic resin that was pre-equilibrated with 50 mM Phosphate buffer at pH 6.3. The desired protein was then eluted using 50 mM Phosphate buffer containing NaCl at pH 6.3.

### EXAMPLE 3

### Chromatography III: Hydrophobic Interaction Chromatography

The eluate at the end of example 2 was loaded onto the hydrophobic interaction chromatography chromatographic (TSK-Butyl 650M) that was pre-equilibrated with 2 M sodium acetate, pH 6.2. The desired protein was then eluted using 0.67 M Sodium Acetate pH 6.0, at a conductivity of 32 mS/cm.

### EXAMPLE 4

### Chromatography IV: Hydrophobic Interaction Chromatography

Alternatively, the eluate from example 1 was loaded onto the hydrophobic interaction chromatography chromatographic resin (TSK-Butyl 650M) that was pre-equilibrated with 425 mM Sodium Citrate, 50 mM Phosphate, pH 6.5 to bind the protein on to the column. The protein was then eluted using 212 mM Sodium Citrate, 50 mM Phosphate, pH 6.5, at a conductivity of 34 mS/cm.

### EXAMPLE 5

### Chromatography V: Anion Exchange Chromatography

The eluate from example 4 was loaded onto the anion exchange chromatographic resin that was pre-equilibrated with Phosphate buffer containing NaCl at pH 6.5. The desired protein was eluted using Phosphate buffer containing NaCl at pH 6.5 and at a conductivity of 12-24 mS/cm.

1. A method of purification of TNFR:Fc fusion protein comprising hydrophobic interaction chromatography performed in bind elute mode wherein the buffer solution used in the said chromatography does not contain any additives.

2. A method of purification of TNFR:Fc fusion protein comprising
a) loading the TNFR:Fc protein containing mixture onto a hydrophobic interaction chromatography resin with a buffer at a pH from about 6.0 to about 7.0,
b) eluting the TNFR:Fc protein from the said resin with an elution buffer at a pH from about 6.0 to about 7.0 and, wherein the buffer solution used for load or elution in the said chromatography does not contain any additives.

3. A method according to claim 2, wherein the pH of the elution buffer is from about 6.0 to about 6.5.

4. A method according to claim 2, wherein the conductivity of the elution buffer is from about 25 mS/cm to about 40 mS/cm.

5. A method according to any of claim 2, wherein the hydrophobic interaction chromatography is preceded by a Protein-A affinity chromatography.

6. A method according to any of claim 2, wherein the hydrophobic interaction chromatography is preceded or followed by an anion exchange chromatography.

7. A method of purification of TNFR:Fc fusion protein comprising steps of;
a. Protein-A chromatography
b. Hydrophobic Interaction chromatography and
c. Anion-exchange chromatography
wherein the buffer solutions used in the chromatographic steps do not contain additives and,
wherein the said hydrophobic interaction chromatography is performed in bind elute mode.

8. A method of purification of TNFR:Fc fusion protein comprising steps of;
a. Protein-A chromatography
b. Anion-exchange chromatography and
c. Hydrophobic Interaction chromatography
wherein the buffer solutions used in the chromatographic steps do not contain additives and,
wherein the said hydrophobic interaction chromatography is performed in bind elute mode.

9.-11. (canceled)
